(19) United States
(12) Patent Application Publication
(10) Pub. No.: US 2010/0087811 A1
(43) Pub. Date: Apr. 8, 2010

(54) Systems and Methods for Controlling Patient Catheters

(75) Inventors: David A. Herrin, Seattle, WA (US); Mark A. Tempel, Sammamish, WA (US); Neil Mcilvaine, Seattle, WA (US)

Correspondence Address: PERKINS COIE LLP;PATENT-SEA, P.O. BOX 1247, SEATTLE, WA, 98111-1247, US

(73) Assignee: CoAptus Medical Corporation, Redmond, WA (US)

(21) Appl. No.: 12/246,349

(22) Filed: Oct. 6, 2008

(51) Int. Cl. A61B 18/18; A61M 25/092

(52) U.S. Cl. 606/40; 604/95.01; 604/510

(57) ABSTRACT

Systems and methods for controlling patient catheters are disclosed. A system in accordance with a particular embodiment includes a catheter carrying multiple active elements, and a controller connected to the catheter. The controller can include a housing having directional indicators, and multiple control elements coupled to the multiple active elements. Individual control elements can be moveable relative to the housing to control the motion of the active elements, and the multiple control elements can be positioned so that manipulation of the multiple control elements in a first order that is clockwise or counterclockwise as identified by the directional indicators moves the multiple active elements in a first manner, and manipulation of the multiple control elements in a second order opposite the first order moves the multiple active elements in a second manner opposite the first manner.

### TECHNICAL FIELD

The present disclosure is directed generally to systems and methods for controlling patient catheters, including catheters used to seal a patient's patent foramen ovale.

### BACKGROUND

The human heart is a complex organ that requires reliable, fluid-tight seals to prevent de-oxygenated blood and other constituents received from the body's tissues from mixing with re-oxygenated blood delivered to the body's tissues. FIG. 1A illustrates a human heart 100 having a right atrium 101, which receives the de-oxygenated blood from the superior vena cava 116 and the inferior vena cava 104. The de-oxygenated blood passes to the right ventricle 103, which pumps the de-oxygenated blood to the lungs via the pulmonary artery 114. Re-oxygenated blood returns from the lungs to the left atrium 102 and is pumped into the left ventricle 105. From the left ventricle 105, the re-oxygenated blood is pumped throughout the body via the aorta 115.

The right atrium 101 and the left atrium 102 are separated by an interatrial septum 106. As shown in FIG. 1B, the interatrial septum 106 includes a primum 107 and a secundum 108. Prior to birth, the primum 107 and the secundum 108 are separated to form an opening (the foramen ovale 109) that allows blood to flow from the right atrium 101 to the left atrium 102 while the fetus receives oxygenated blood from the mother. After birth, the primum 107 normally seals against the secundum 108 and forms an oval-shaped depression, i.e., a fossa ovalis 110.

In some infants, the primum 107 never completely seals with the secundum 108, as shown in cross-sectional view in FIG. 1C and in a left side view in FIG. 1D. In these instances, a patency often having the shape of a tunnel 112 forms between the primum 107 and the secundum 108. This patency is typically referred to as a patent foramen ovale or PFO 113. In most circumstances, the PFO 113 will remain functionally closed and blood will not tend to flow through the PFO 113, due to the normally higher pressures in the left atrium 102 that secure the primum 107 against the secundum 108. Nevertheless, during physical exertion or other instances when pressures are greater in the right atrium 101 than in the left atrium 102, blood can inappropriately pass directly from the right atrium 101 to the left atrium 102 and can carry with it clots, gas bubbles, or other vaso-active substances. Such constituents in the atrial system can pose serious health risks including hemodynamic problems, cryptogenic strokes, venous-to-atrial gas embolisms, migraines, and in some cases even death.

Traditionally, open chest surgery was required to suture or ligate a PFO 113. However, these procedures carry high attendant risks, such as postoperative infection, long patient recovery, and significant patient discomfort and trauma. Accordingly, less invasive techniques have been developed. Most such techniques include using transcatheter implantation of various mechanical devices to close the PFO 113. Such devices include the Cardia® PFO Closure Device, Amplatzer® PFO Occluder, and CardioSEAL® Septal Occlusion Device. One potential drawback with these devices is that they may not be well suited for the long, tunnel-like shape of the PFO 113. As a result, the implanted mechanical devices may become deformed or distorted and in some cases may fail, migrate, or even dislodge. Furthermore, these devices can irritate the cardiac tissue at or near the implantation site, which in turn can potentially cause thromboembolic events, palpitations, and arrhythmias. Other reported complications include weakening, erosion, and tearing of the cardiac tissues around the implanted devices.

Another potential drawback with the implanted mechanical devices described above is that, in order to be completely effective, the tissue around the devices must endothelize once the devices are implanted. The endothelization process can be gradual and can accordingly take several months or more to occur. Accordingly, the foregoing techniques do not immediately solve the problems caused by the PFO 113.

Still another drawback associated with the foregoing techniques is that they can be technically complicated and cumbersome. Accordingly, the techniques may require multiple attempts before the mechanical device is appropriately positioned and implanted. As a result, implanting these devices may require long procedure times during which the patient must be kept under conscious sedation, which can pose further risks to the patient.

### BRIEF DESCRIPTION OF THE DRAWINGS

FIGS. 1A-1D illustrate a human heart having a patent foramen ovale (PFO) in accordance with the prior art.

FIG. 2A illustrates a catheter positioned proximate to a PFO for treatment in accordance with several embodiments of the disclosure.

FIGS. 2B-2C illustrate catheter controllers configured in accordance with embodiments of the disclosure.

FIGS. 3A-3J illustrate a process for closing a PFO, along with corresponding changes in the configuration of a catheter controller in accordance with an embodiment of the disclosure.

FIG. 4 is a partially schematic, isometric illustration of the interior of a catheter controller configured in accordance with an embodiment of the disclosure.

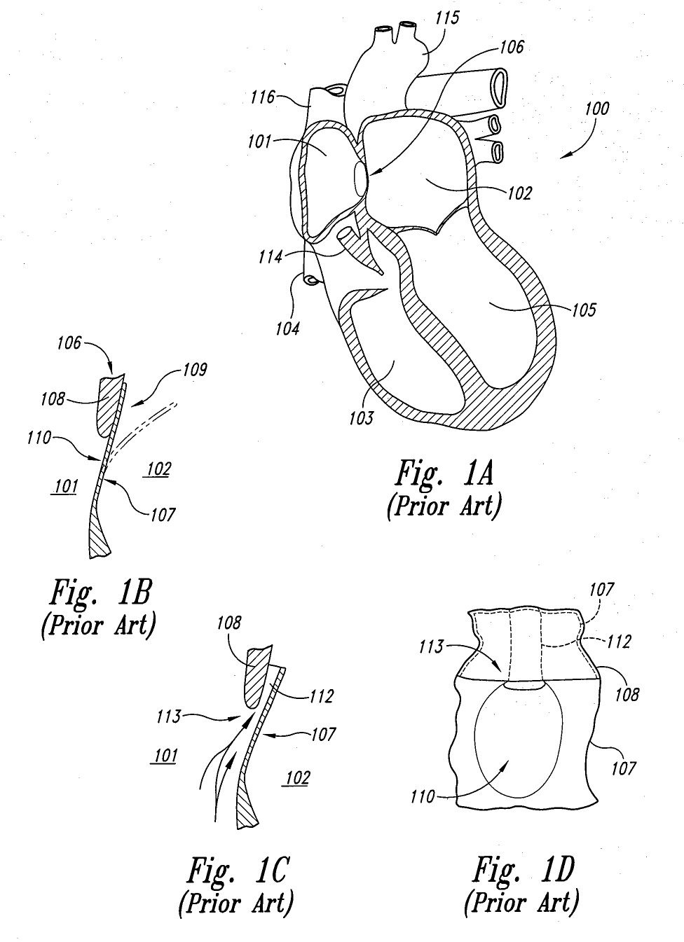

### DETAILED DESCRIPTION

### A. Introduction

Aspects of the present disclosure are directed generally to methods and devices for drawing portions of cardiovascular tissue together, sealing the portions to each other, and controlling the performance of these tasks. Much of the discussion below is provided in the context of sealing patent foremen ovales (PFOs). However, in other embodiments, these techniques may be used to treat other types of cardiac tissue and/or tissue defects. The energy to seal the PFO is generally provided by an energy transmitter. For purposes of discussion, much of the following description is provided in the context of energy transmitters that include electrodes configured to seal cardiac tissue by delivering radio frequency (RF) energy. In other embodiments, the energy transmitters can have other arrangements and can deliver other types of energy, for example, microwave energy, laser energy, or ultrasound energy.

In general, many of the techniques and associated devices described below include advancing a catheter into the right atrium of the patient's heart, piercing the septum between the right atrium and the left atrium, and placing an electrode or other energy transmitter in the left atrium. The energy transmitter applies energy to the septum to seal the PFO, and is then drawn back through the septum. The catheter can then be withdrawn from the patient's body, leaving no foreign objects behind. A residual hole in the septum remaining after the electrode is withdrawn from the left atrium to the right atrium is expected to close over a short period of time as a result of the body's natural healing response.

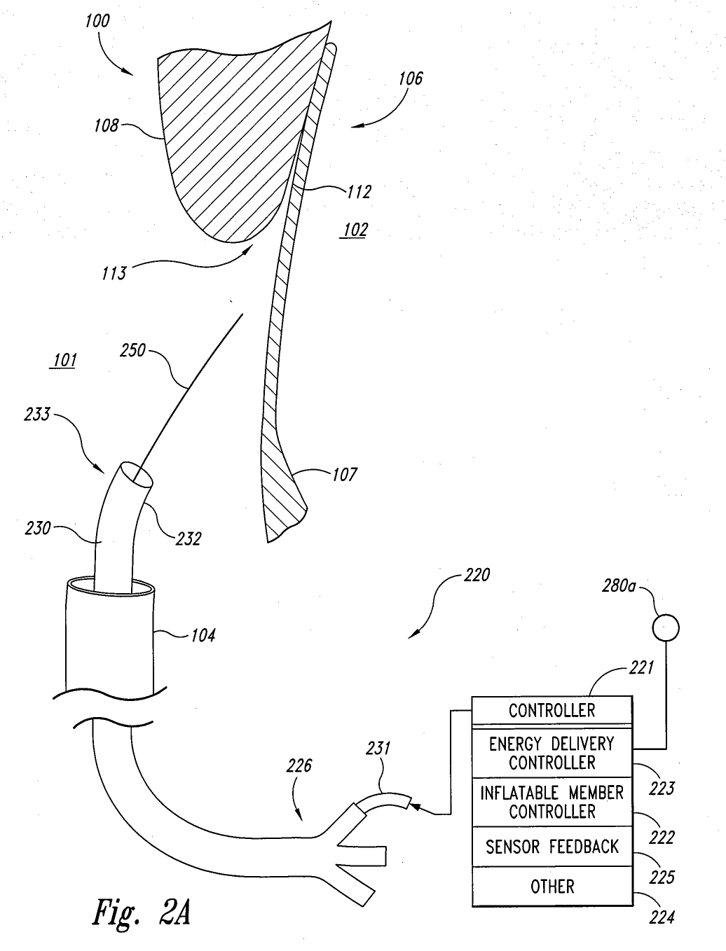

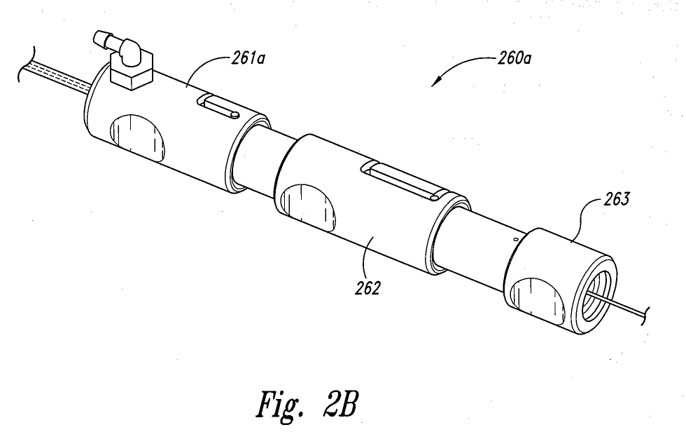

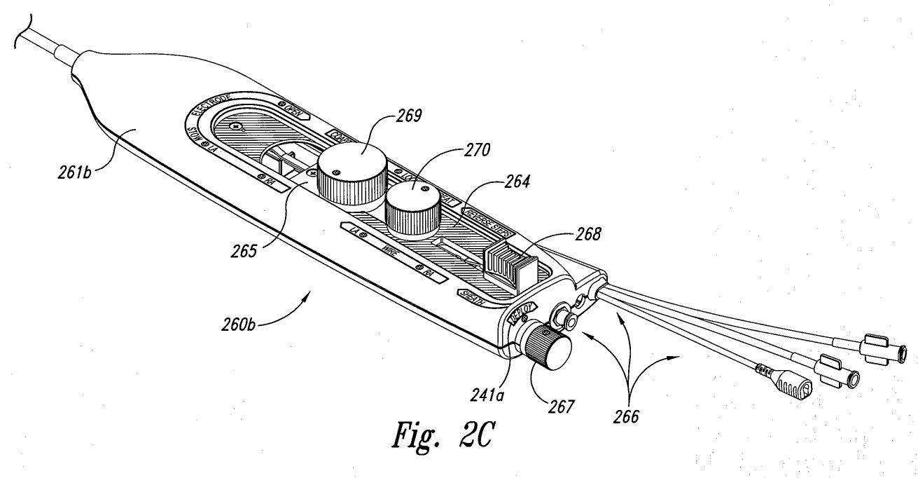

Several details describing devices or processes that are well-known to those of ordinary skill in the relevant art and often associated with such devices and processes are not set forth in the following description for purposes of brevity. Those of ordinary skill in the relevant art will understand that further embodiments may include features not disclosed in the following sections, and/or may eliminate some of the features described below with reference to FIGS. 2A-4. Certain elements in the following description are referred to as “first,” “second,” etc., but such elements may be referred to by different numerical identifiers, or no numerical identifiers, in the claims.

FIG. 2A is a schematic, not-to-scale illustration of the general components of a system 220 used to treat a patient in accordance with several embodiments of the disclosure. The system 220 generally includes one or more patient treatment devices, a term which, as used herein, includes devices that provide direct therapeutic benefits, and/or associated functions, including but not limited to, diagnostic functions, feedback functions, and/or positioning functions. The system 220 can include one or more guidewires 250 that are directed into the patient via an introducer 226, and are then threaded through the patient's vascular system to the heart 100. In the illustrated embodiment, the guidewire 250 enters the right atrium 101 from the inferior vena cava 104, and in other embodiments, the guidewire 250 can enter the right atrium 101 or other heart chamber from other vessels. One or more guidewires may also pass into the left atrium 102. One or more catheters 230 are then threaded along the guidewire 250 via corresponding lumens to treat a PFO 113 (e.g., the PFO tunnel 112) located between the primum 107 and the secundum 108 of the patient's septum 106. The catheter lumen(s) can be flushed with saline, contrast agent, and/or another appropriate biocompatible fluid, either continuously or at selected intervals, to prevent clot formation, enhance visualization, and/or lubricate the relative motion between the catheter(s) and devices within the lumens.

The catheter 230 typically includes a distal end 232 within the patient's body, a working portion 233 toward the distal end 232, and a proximal end 231 that extends outside the patient's body. A controller 221 controls the functions carried out by the catheter 230 and the rest of the system 220, and can include an energy delivery controller 223 to control RF or other energy transmitted to the patient, an inflatable member controller 222 to control the operation of one or more (optional) inflatable members in the patient, a sensor feedback unit 225 to receive diagnostic information, and other controllers 224 to control other functions, for example, the motion of various guidewires and/or other elements of the system 220, and/or fluid delivery to elements of the system 220. When the energy transmitter or delivery device includes an electrode, it may be operated in a monopolar manner, in which case a return electrode 280a is located remotely from the PFO 113. For example, the return electrode 280a can include a patient pad located at the back of the patient's left shoulder. In other embodiments, the electrode can operate in a bipolar manner, in which case the return electrode is generally located at or close to the PFO 113.

FIGS. 2B and 2C illustrate representative controllers in accordance with particular embodiments of the disclosure. FIG. 2B illustrates a first controller 260a configured to control the operation of a self-centering guidewire that is used to position components of the overall system 200 described above within the patient's heart. The first controller 260a can include a housing 261a that in turn carries a deployment knob 262 and a connector knob 263. The deployment knob 262 is used to deploy the self-centering guidewire, and the connector knob 263 is used to disengage a connector of the self-centering guidewire so that the self-centering guidewire can be removed from the patient. Further details of the first controller 260a and associated self-centering guidewires are included in co-pending U.S. application Ser. No. ______ (Attorney Docket No. 57120.8016US1), filed concurrently herewith and incorporated herein by reference.

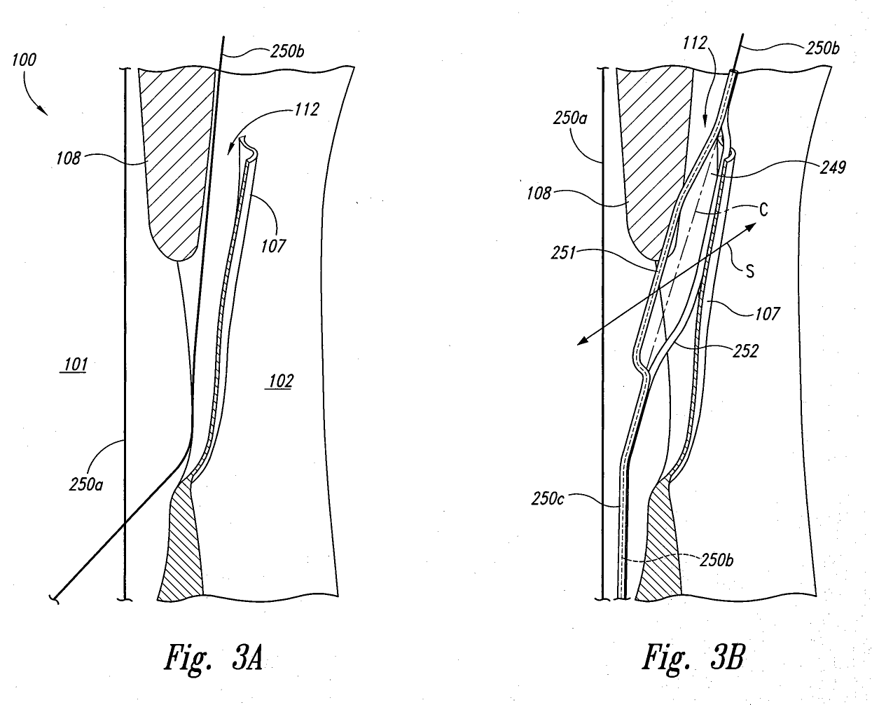

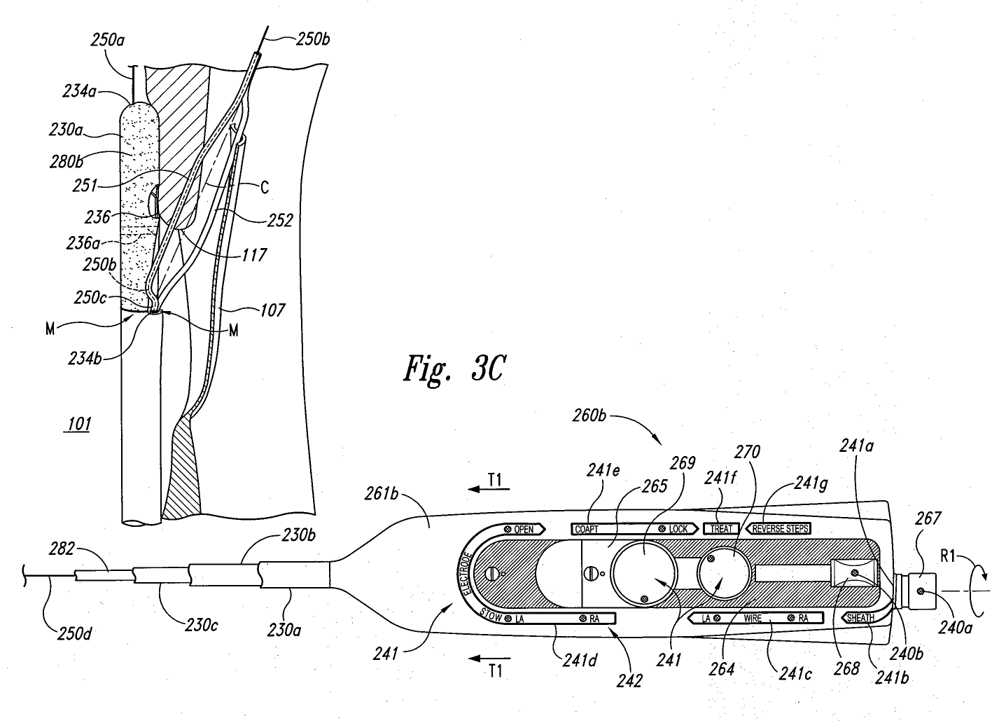

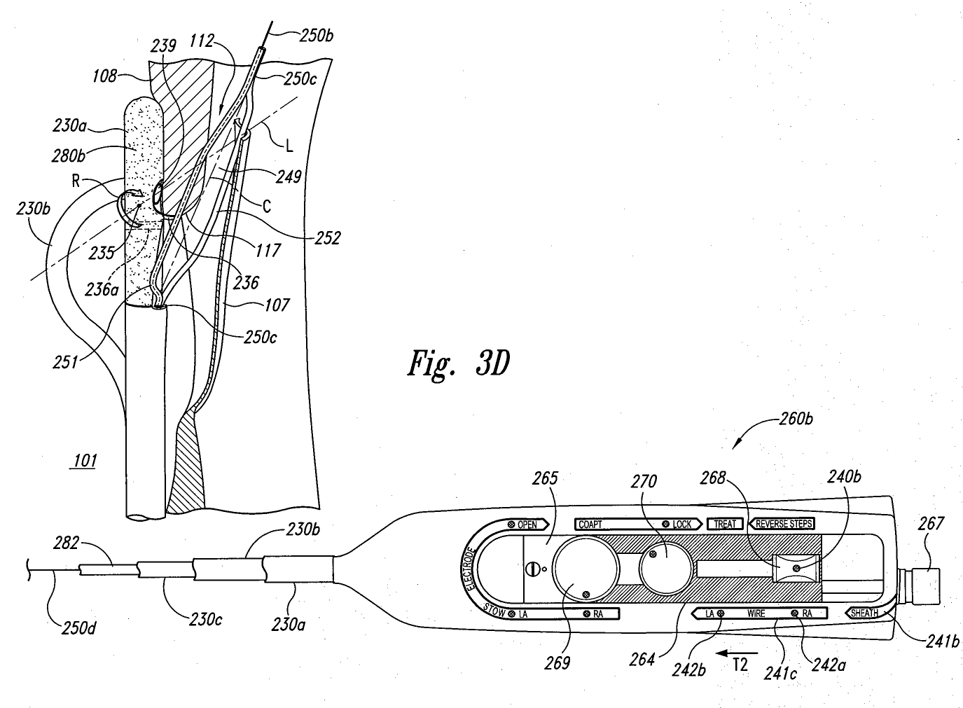

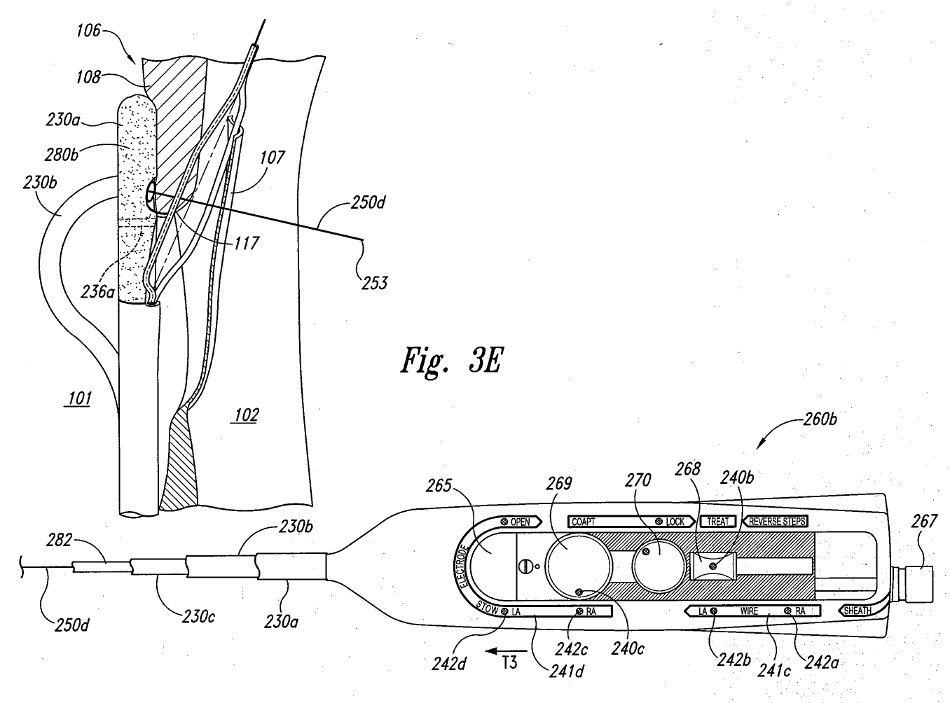

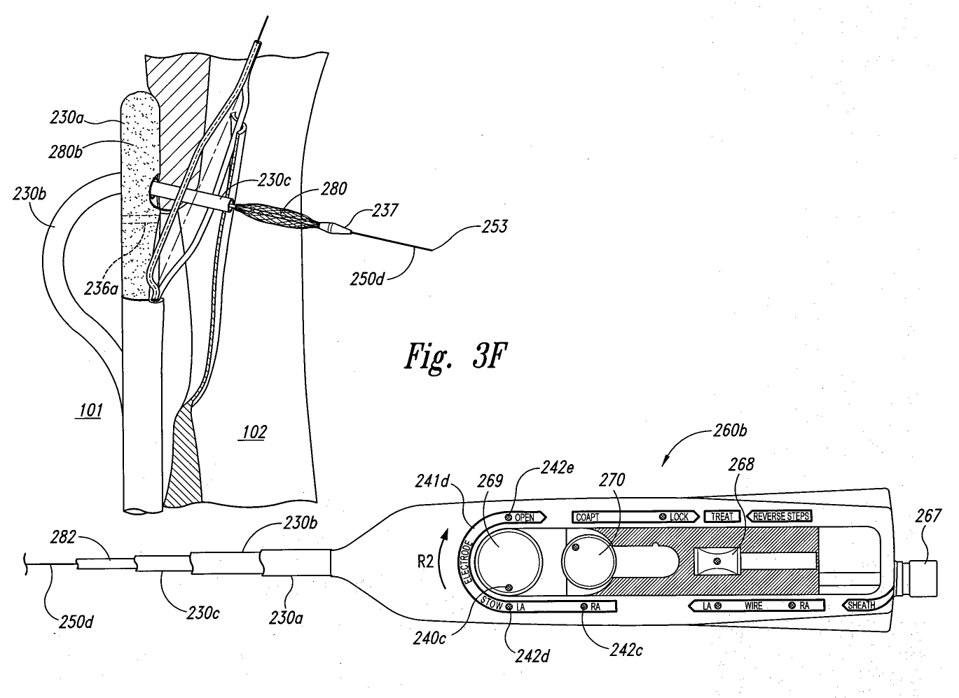

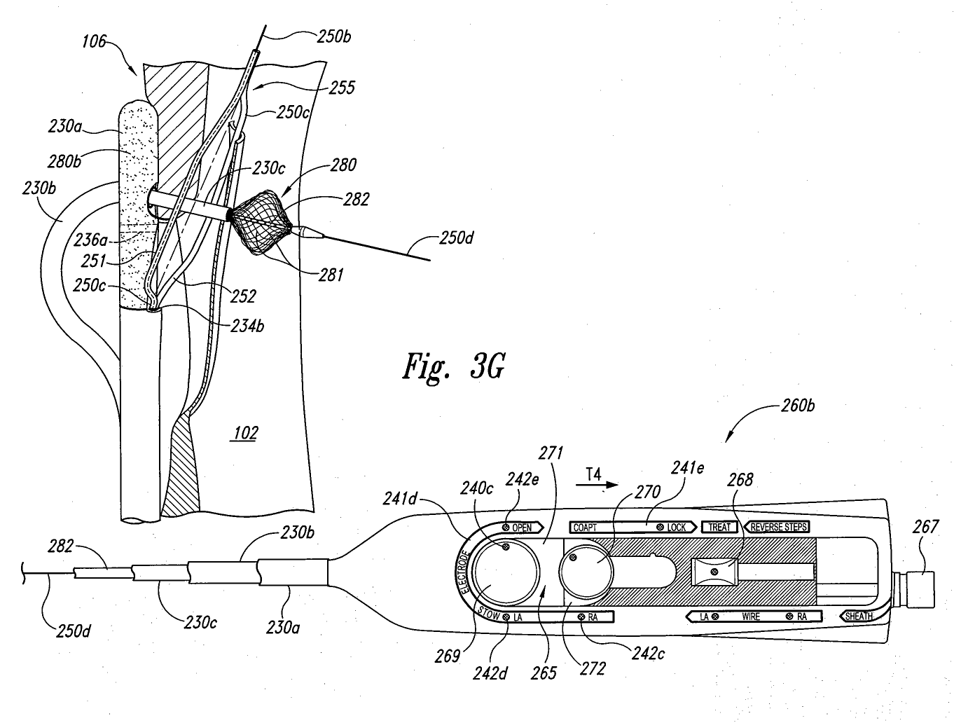

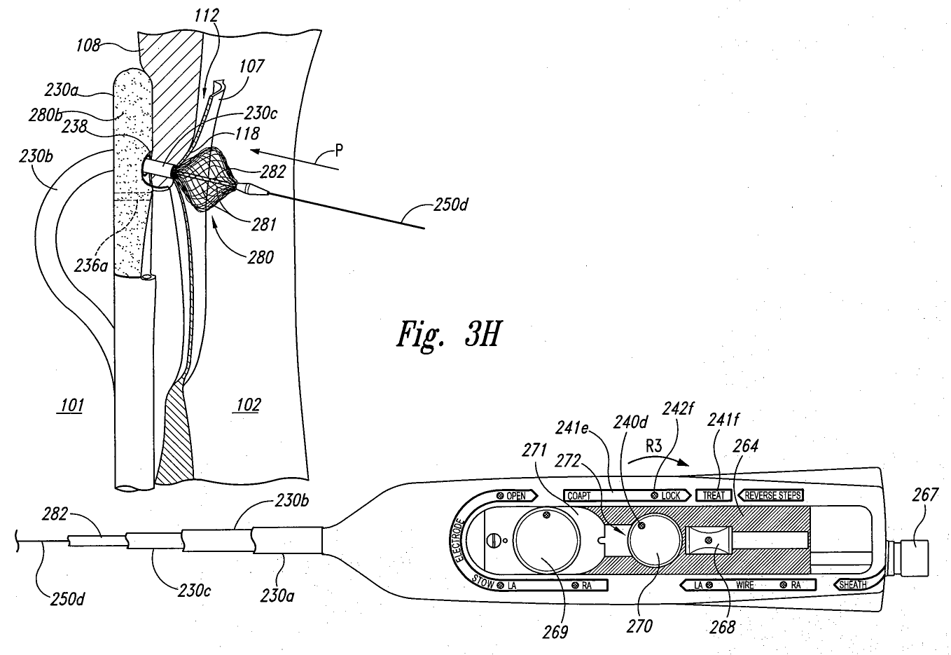

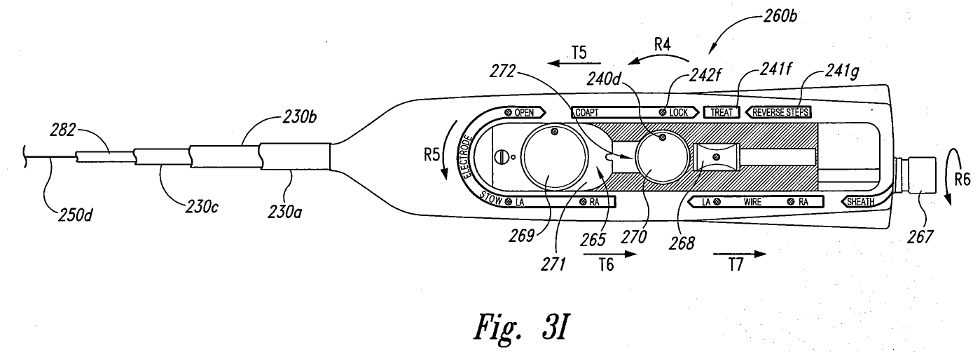

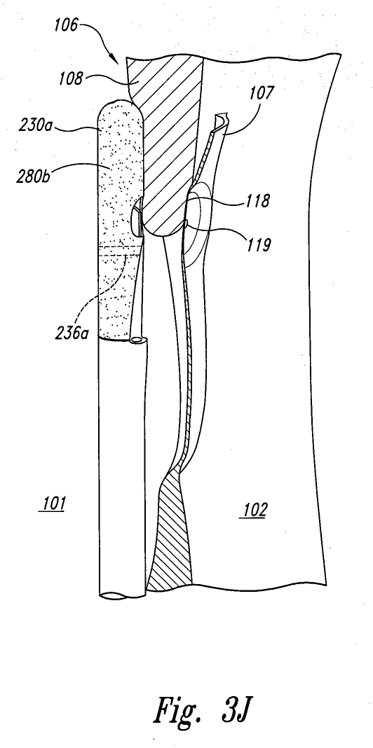

FIG. 2C is a partially schematic, isometric illustration of a second controller 260b that can be used alone or in conjunction with the first controller 260a (FIG. 2B) to control other aspects of the overall system 200 described above with reference to FIG. 2A. The second controller 260b can include a housing 261b that carries multiple controls or control elements. In a particular embodiment shown in FIG. 2C, the control elements can include a catheter bend control 267, a penetrating guidewire control 268, an electrode deployment control 269, and a coaption control 270. The penetrating guidewire control 268 can be carried by a first carriage 264, and the electrode deployment control 269 and coaption control 270 can be carried by a second carriage 265. The two carriages 264, 265 can be moved together or independently, depending upon the particular operation the practitioner executes with the controls 267-270. The housing 261b can also include one or more ports 266 that supply electrical power, flow visualization fluid, saline, other guidewires, and/or other implements or materials, depending on embodiment details. The overall operation of the second controller 260b is now described below with reference to FIGS. 3A-3J.

### B. Techniques and Systems for Treating a PFO

FIGS. 3A-3J include enlarged cross-sectional views of the heart regions around a PFO, and illustrate representative techniques and associated devices for sealing the PFO in accordance with a particular embodiment. Beginning with FIG. 3A, a practitioner passes a right atrial guidewire 250a into the right atrium 101 of the patient's heart 100. Optionally, the practitioner can continue to advance the right atrial guidewire 250a into the superior vena cava. The practitioner then passes a left atrial guidewire 250b into the right atrium 101, through the PFO tunnel 112 and into the left atrium 102. Accordingly, the left atrial guidewire 250b is positioned in the tunnel 112 between the primum 107 and the secundum 108. Suitable imaging processes (e.g., transthoracic ultrasound or TTE, intra-cardiac echo or ICE, transesophageal echo or TEE, fluoroscopy, and/or others) known to those of ordinary skill in the relevant art may be used to position the guidewires 250a, 250b and/or other devices used during the procedure.

In another embodiment, the left atrial guidewire 250b is routed as described above, but before the right atrial guidewire 250a is introduced. The right atrial guidewire 250a is instead pre-loaded into a delivery catheter (described later with reference to FIG. 3C), and the delivery catheter, with the right atrial guidewire 250a on board, is threaded along the left atrial guidewire 250b to the right atrium 101 (e.g., at or near the junction between the right atrium 101 and the inferior vena cava). Once the delivery catheter is in the right atrium 101, the right atrial guidewire 250a can be deployed to the location shown in FIG. 3A.

In FIG. 3B, the practitioner has threaded a self-centering guidewire 250c along the left atrial guidewire 250b and into the tunnel 112. Alternatively, the self-centering guidewire 250c can be pre-loaded into the delivery catheter (described later with reference to FIG. 3C) and both can be advanced together along the left atrial guidewire 250b. This latter arrangement, e.g., in combination with pre-loading the right atrial guidewire 250a as described above, can prevent the left atrial guidewire 250b and the right atrial guidewire 250a from becoming twisted. In either embodiment, the self-centering guidewire 250c can include a first branch 251 and a second branch 252 positioned around an enclosed region 249. In a particular aspect of this embodiment, the first branch 251 is hollow so as to receive the left atrial guidewire 250b along which the self-centering guidewire 250c is passed. The first and second branches 251, 252 can be at least somewhat compliant and resilient and can accordingly spread or tighten the primum 107 laterally, as indicated by arrow S, upon being introduced into the tunnel 112. By stretching the primum 107, the self-centering guidewire 250c can draw the primum 107 toward the secundum 108. In addition, the branches 251, 252 can be symmetric relative to a central axis C and can accordingly center the self-centering guidewire 250c within the PFO tunnel 112. Furthermore, the closed shape provided by the first and second branches 251, 252 can provide the guidewire 250c with a degree of lateral rigidity along the axis identified by arrow S. Accordingly, when the guidewire 250c is placed in the tunnel 112, the resilience provided by the primum 107 and/or the secundum 108 can force the guidewire 250c to assume the orientation shown in FIG. 3B, e.g., with the generally flat plane of the enclosed region 249 “sandwiched” between and facing toward the primum 107 on one side and the secundum 108 on the other. The lateral rigidity of the self-centering guidewire 250c when it is deployed can also prevent it from twisting, which in turn can make it easier for the practitioner to accurately seal the tunnel 112.

Turning next to FIG. 3C, the practitioner has threaded a delivery catheter 230a along the right atrial guidewire 250a and the self-centering guidewire 250c, which is in turn threaded along the left atrial guidewire 250b, as discussed above. Or, as discussed above, the right atrial guidewire 250a and the self-centering guidewire 250c can be pre-loaded into the delivery catheter 230a and deployed once the delivery catheter 230a has been threaded along the left atrial guidewire 250b until it is positioned in the right atrium 101. In either embodiment, the delivery catheter 230a can include a right atrial guidewire opening 234a that receives the right atrial guidewire 250a, and a left atrial guidewire opening 234b that receives the self-centering guidewire 250c and the left atrial guidewire 250b over which the self-centering guidewire 250c is passed. In this embodiment, the self-centering guidewire 250c has a generally elliptical cross-sectional shape, and accordingly, the left atrial guidewire opening 234b has a similar shape. With this arrangement, the self-centering guidewire 250c is “keyed” to the delivery catheter 230a. Accordingly, the delivery catheter 230a has a known orientation relative to the self-centering guidewire 250c when the delivery catheter 230a reaches the position shown in FIG. 3C. The upward progress of the delivery catheter 230a can be limited by a “tree crotch effect” provided by the delivery catheter 230a positioned on one side of the septal limbus 117, and the combined left atrial guidewire 250b and self-centering guidewire 250c on the other side of the limbus 117. In addition, radiopaque markers M can be located at the left atrial guidewire opening 234b and the point at which the branches 251, 252 bifurcate. In a particular embodiment, the markers M can therefore be co-located or nearly co-located when the delivery catheter 230a has been properly advanced. Once the delivery catheter 230a has the position shown in FIG. 3C, the right atrial guidewire 250a can optionally be withdrawn, or it can remain in place for additional steps, including for the remainder of the procedure.

As noted above with reference to FIG. 2, the overall system can include a return electrode positioned close to the PFO. FIG. 3C illustrates a return electrode 280b carried by the delivery catheter 230a so as to operate in a bipolar manner with an electrode delivered in accordance with an embodiment of the disclosure. In a particular aspect of this embodiment, the return electrode 280b can include an electrically conductive coating or sleeve positioned at the outside of the delivery catheter 230a, and coupled to an electrical return terminal (e.g., at the controller 221 shown in FIG. 2) via a lead wire (not visible in FIG. 3C). In another embodiment, the return electrode 280b can have other arrangements and/or configurations in which it is positioned close to the primum 107 and/or the secundum 108.

FIG. 3C also illustrates the second controller 260b, to which the delivery catheter 230a is connected. The delivery catheter 230a houses a positioning catheter 230b, an electrode delivery catheter 230c, an actuator 282, and a penetrating guidewire 250d. The catheter bend control 267 can control the manner in which the positioning catheter 230b bends, and the penetrating guidewire control 268 can control the motion of the penetrating guidewire 250d. The electrode deployment control 269 and the coaption control 270 can control the operation of the electrode delivery catheter 230c and the actuator 282. In other embodiments, the delivery catheter 230c can include active elements other than those described above, and the second controller 260b can include other corresponding controls or control elements.

The illustrated controller 260b includes, in addition to the controls 267-270 described above, a plurality of directional indicators 241. The directional indicators 241 can be arranged in an order and sequence that corresponds to the order and sequence with which a practitioner carries out subsequent processes for sealing the patient's PFO. In a particular embodiment shown in FIG. 3C, the directional indicators 241 include a first directional control indicator 241a (also shown in FIG. 2C) positioned proximate to the catheter bend control 267, a second directional indicator 241b positioned at the lower right hand corner of the housing 261b, a third directional indicator 241c positioned along the lower surface of the housing 261b, and a fourth directional indicator 241d that wraps around the control elements in a clockwise direction from the lower region of the housing 261b to an upper region of the housing 261b. A fifth directional indicator 241e is positioned along the upper portion of the housing, a sixth directional indicator 241f is positioned adjacent to the fifth directional indicator 241e, and a seventh directional indicator 241g is positioned toward the upper left region of the housing 261b. Several of the directional indicators 241 can include arrowheads that direct the practitioner to follow a clockwise path as the practitioner manipulates the control elements. The seventh directional indicator 241g can have an arrowhead pointing in the reverse direction, indicating the order in which the practitioner will manipulate the control elements once the tissue sealing process has been completed. Accordingly, the directional indicators 241 can provide a graphical, intuitive, ordered sequence to aid the practitioner in carrying out the series of steps used to seal the PFO.

The controller 260b can also include control element indicators 240 carried by the control elements 267-270, and corresponding housing indicators 242 carried by the housing 261b. In a particular aspect of embodiments shown in FIG. 3C and the following Figures, many of the control element indicators 240 can align with corresponding housing indicators 242 only when the control elements on which the control element indicators 240 are positioned are ready to be operated. Further details of this arrangement will become apparent from the following discussion.

With the controller 260b and the delivery catheter 230a in the respective positions shown in FIG. 3C, the practitioner can rotate the catheter bend control to 267 clockwise as indicated by R1. The practitioner can be prompted to take this action by noting that the catheter bend control 267 is at the far right side of the housing 261b and therefore at the beginning of the sequence of arrows that progress in a clockwise direction. The catheter bend control 267 can include a first control element indicator 240a that is aligned with a corresponding housing indicator on the right-facing side of the housing 261b (not visible in FIG. 3C). This alignment can also prompt the practitioner to manipulate the catheter bend control 267. As the practitioner rotates the catheter bend control 267 clockwise as indicated by R1, the first carriage 264 and the second carriage 265 move together as a unit and advance the positioning catheter 230b in a distal direction relative to the delivery catheter 230a. FIG. 3D illustrates the result of this action at the patient's heart.

As shown in FIG. 3D, the positioning catheter 230b is now deployed from the delivery catheter 230a in the right atrium 101. In this embodiment, the positioning catheter 230b is deployed by applying an axial force to it, causing it to buckle or bend outwardly through a corresponding slot (not visible in FIG. 3D) in the outer surface of the delivery catheter 230a. Accordingly, the positioning catheter 230b can assume the shape shown in FIG. 3D. In one arrangement, the distal end of the positioning catheter 230b is eccentrically connected to a pivot axle 235, which allows the positioning catheter 230b to rotate as indicated by arrow R as it buckles. As the positioning catheter 230b rotates, it can position the exit opening of a lumen 239 to face outwardly from the delivery catheter 230a.

The lumen 239 can also face directly toward the secundum 108, and can be aligned with the central axis C above the limbus 117, as a result of the features of the self-centering guidewire 250c, the delivery catheter 230a and the positioning catheter 230b. In particular, the self-centering guidewire 250c can be centered within the tunnel 112, with the plane defined by the enclosed region 249 facing directly toward the secundum 108.

Because the illustrated self-centering guidewire 250c has a generally flat shape (and can optionally stretch the primum 107), the primum 107 and the secundum 108 can tend to keep the self-centering guidewire 250c from rotating or twisting about its lengthwise axis relative to the tunnel 112. In addition, the branches 251, 252 of the self-centering guidewire 250 can be secured relative to each other in a manner that resists twisting. Because the self-centering guidewire 250c is keyed with the delivery catheter 230a, as discussed above with reference to FIG. 3C, the delivery catheter 230a is prevented or at least restricted from rotating about its lengthwise axis relative to the tunnel 112. Accordingly, when the positioning catheter 230b is deployed, the lumen 239 can face directly toward the secundum 108, e.g., at an orientation of from about 80° to about 135°, and in a particular embodiment, about 105°. It is expected that in at least some embodiments, an orientation of about 105° results in a subsequent tissue penetration operation that effectively penetrates the secundum 108 and the primum 107 with a reduced likelihood for penetrating other tissue in the left atrium. In addition, this orientation can increase the likelihood of penetrating the primum 107, e.g., when the tunnel 112 is relatively short. The lumen 239 can also be located at the lateral center or approximate center of the tunnel 112 (e.g., measured laterally along a lateral axis L that is generally transverse to the central axis C). The “tree-crotch effect” described above can act to locate the lumen 239 above the limbus 117, but not so high that the lumen 239 is above the primum 107.

In a particular embodiment, a limbus stop 236 is connected to the positioning catheter 230b. As the positioning catheter 230b rotates, the limbus stop 236 rotates outwardly to the position shown in FIG. 3D. When the practitioner applies an axial (e.g., upward) force to the delivery catheter 230a, the limbus stop 236 can nudge up against the limbus 117. In other embodiments, the limbus stop 236 can be eliminated. In still further embodiments, the delivery catheter 230b can include a limbus marker 236a, in addition to or in lieu of the limbus stop 236. The limbus marker 236a can be a pin or other element made from gold, platinum or another radiopaque material. The limbus marker 236a can help guide the operator to correctly position the delivery catheter 230a relative to the limbus 117 before penetrating the secundum 108. The limbus 117 itself may be illuminated with a contrast agent. In many cases, the delivery catheter 230a and other components illustrated in FIG. 3D may be formed from plastics or other materials that do not readily appear during fluoroscopy processes. Accordingly, the limbus marker 236a can provide a readily visible locater on the delivery catheter 230a to aid the practitioner during a tissue sealing procedure. The limbus marker 236a can be positioned at a known location along the length of the delivery catheter 230a, for example 4 mm below the axis along which a penetrating guidewire is deployed. Further details of the penetrating guidewire are described later with reference to FIG. 3E.

As shown in FIG. 3D, and as a result of the practitioner rotating the catheter bend control 267 to advance the delivery catheter 230b as described above, a second control element indicator 240b carried by the penetrating guidewire control 268 aligns with a first housing indicator 242a carried by the housing 261. This, in combination with the arrowhead provided by the second directional indicator 241b, directs the practitioner to the penetrating guidewire control 268 and the third directional indicator 241c. In a particular embodiment, the third directional indicator 241c is also labeled “WIRE”, with the first housing indicator 242a labeled “RA” identifying the patient's right atrium. The practitioner can slide the penetrating guidewire control 268 from right to left as indicated by the third directional indicator 241c to align the second control element indicator 240b with the next housing indicator, e.g., the second housing indicator 242b, which is labeled “LA” for left atrium. FIG. 3E illustrates the result at the distal end of the delivery catheter 230a.

As shown in FIG. 3E, the penetrating guidewire 250d or other penetrating device or member is now deployed from the positioning catheter 230b. The penetrating guidewire 250d can include a penetrating tip 253 that penetrates through the secundum 108 and the primum 107, so as to cross the entire septum 106 into the left atrium 102. In a particular embodiment, the penetrating tip 253 can include an RF electrode that is advanced through the septum 106 in a stepwise fashion or in a continuous fashion, as disclosed in U.S. application Ser. No. _______ (Attorney Docket No. 57120.8016US1). The electrode can have a generally spherical or ball-type shape, with a diameter of up to about 1.0 mm. In other embodiments, the penetrating tip 253 can have other shapes or configurations, and/or can be advanced using other techniques, and/or can employ other non-RF methods for penetrating the septum 106. Such configurations include, but are not limited to a penetrating tip 253 having a sharp distal end that pierces the septum 106. For example, the penetrating tip can include one or more razor-like elements or blades that score the septum 106. The blades can deploy laterally outwardly, and/or can be deployed from an inflatable balloon. In other embodiments, the tip 253 can include rotoblades, laser energy emitters, and/or ultrasound energy emitters.

After the practitioner has moved the penetrating guidewire control 268 to the position shown in FIG. 3E, the practitioner's attention is next directed to the electrode deployment control 269, by virtue of the arrowhead at the end of the third directional indicator 241c, and by virtue of the alignment between a third control element indicator 240c carried by the electrode deployment control 269 and a corresponding third housing indicator 242c labeled “RA” for right atrium. The third housing indicator 242c is located at the fourth directional indicator 241d, labeled “ELECTRODE.” The practitioner skips over the coaption control 270 (for now) because the coaption control 270 does not have a directional indicator aligned with a corresponding housing indicator. The practitioner slides the electrode deployment control 269 from right to left, moving the second carriage 265 along with the coaption control 270 in the direction indicated by arrow T3. This action also moves the third control element indicator 240c from alignment with the third housing indicator 242c to alignment with a fourth housing indicator 242d labeled “LA” for left atrium. This action indicates that the practitioner is deploying the electrode across the patient's septum from the right atrium to the left atrium, as described below with reference to FIG. 3F.

In FIG. 3F, the practitioner has moved the electrode deployment control 269 as described above with reference to FIG. 3E to advance the electrode catheter 230c along the penetrating guidewire 250d from the right atrium 101 into the left atrium 102. The electrode catheter 230c can include a dilator 237 that temporarily stretches the hole initially created by the penetrating guidewire 250d to allow additional components to pass into the left atrium 102. These components can include an electrode device 280 and an optional inflatable member (not shown in FIG. 3F). In a particular embodiment, the penetrating guidewire 250d can form a hole having a diameter of about one millimeter, and the dilator 237 can have a diameter of about two millimeters to temporarily stretch the hole to a diameter of about two millimeters. When the electrode catheter 230c and the penetrating guidewire 250d are later withdrawn, the hole can relax back to a diameter of about one millimeter. In other embodiments, these dimensions can have other values. In any of these embodiments, the dilator 237 and/or the penetrating tip 253 can include radiopaque markings for enhanced visibility during fluoroscopic visualization.

With the second controller 260b in the configuration shown in FIG. 3F, the third control element indicator 240c is aligned with the fourth housing indicator 242d. The practitioner follows the clockwise path of the fourth directional indicator 241d and rotates the electrode deployment control 269 clockwise as indicated by arrow R2 to align the third control element indicator 240c with a fifth housing indicator 242e. As indicated by the text legend at the fourth directional indicator 241d, this action changes the configuration of the electrode from a “STOW”ed or collapsed configuration to an “OPEN” or expanded configuration, as shown and described below with reference to FIG. 3G.

In FIG. 3G, the practitioner has deployed the electrode device 280 in the left atrium 102 by rotating the electrode deployment control 269 as indicated above with reference to FIG. 3F. In a particular embodiment, the electrode device 280 includes a braided arrangement of electrically conductive filaments 281 connected at a proximal end to the electrode catheter 230c, and at a distal end to the actuator tube 282. As the electrode deployment control 269 is rotated to the position shown in FIG. 3G, the actuator tube 282 moves proximally to expand or open the electrode device 280. Accordingly, the electrode device 280 can have a spheroid or (more generally) an ellipsoid shape.

Prior to engaging the electrode device 280 with the septum 106, the practitioner can withdraw the self-centering guidewire 250c and the left atrial guidewire 250b by separating or opening the first and second branches 251, 252 at a separation location 255, allowing them to pass downwardly around opposite sides of the electrode catheter 230c and into the left atrial guidewire opening 234b. Further details of embodiments for performing this task are described in U.S. application Ser. No. ______ (Attorney Docket No. 57120.8016US1) previously incorporated by reference.

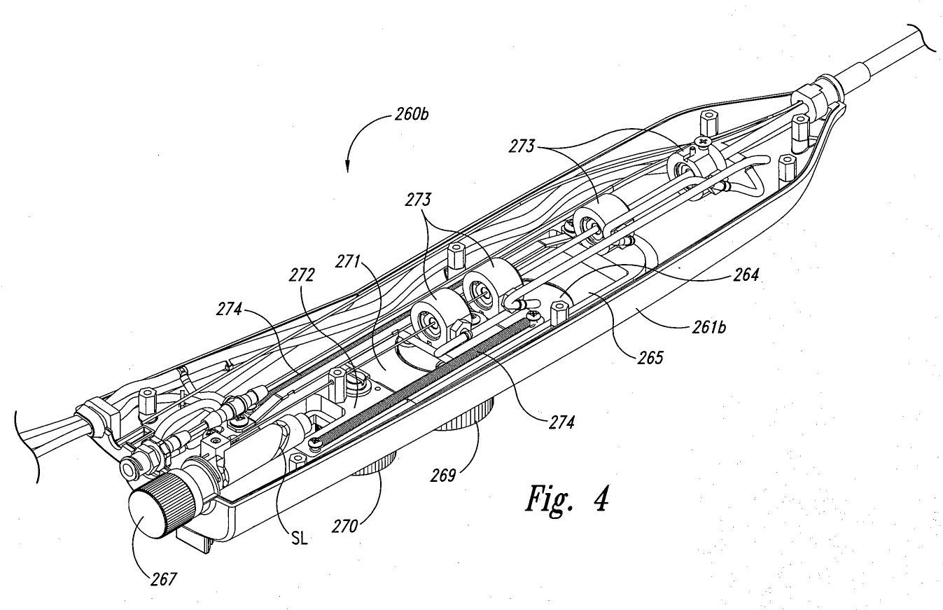

With the second controller 260b in the configuration shown in FIG. 3G, the practitioner again follows the clockwise direction provided by the fourth and fifth directional indicators 241d-241e and slides the coaption control 270 from left to right, as indicated by arrow T4. As the practitioner moves the coaption control 270 in this manner, two portions of the second carriage 265 (shown as a first portion 271 and a second portion 272) can separate from each other. In particular, the second portion 272 can separate from the first portion 271 as it moves along with the coaption control 270 from left to right. The first portion 271 can remain in place, or (more typically), the first portion 271 can move from left to right, but not by as much as does the second portion 272. The two portions 271, 272 can be forced toward each other via springs, described later with reference to FIG. 4. The ability of the two portions 271, 272 to separate from each other and yet be forced toward each other allows the electrode device 280 to compress the primum 107 and the secundum 108 toward the delivery catheter 230a. FIG. 3H illustrates the result of this action at the patient's heart.

In FIG. 3H, the practitioner has removed the self-centering guidewire 250c (FIG. 3G) and the left atrial guidewire 250b (FIG. 3G), and, (by manipulating the coaption control 270 as described above) has applied an axial force to the electrode catheter 230c in a generally proximal direction P. The axial force draws the electrode device 280 snugly up against the primum 107. This force can also clamp the primum 107 against the secundum 108, and can clamp both the primum 107 and the secundum 108 between the electrode device 280 and a backstop surface 238. In an embodiment shown in FIG. 3H, the backstop surface 238 includes the outwardly facing, conductive external surface of the delivery catheter 230a, e.g., the return electrode 280b. Accordingly, the electrode device 280 can operate in a bipolar manner via the return electrode 280b. In other embodiments, the backstop surface 238 can have other locations and/or arrangements. For example, the backstop surface 238 can be separate from the delivery catheter 230a, and/or can be electrically non-conductive, so that the electrode device 280 operates in a monopolar manner.

Once the septal tissue has been clamped, the practitioner locks the coaption control 270 in place by rotating the coaption control 270 clockwise, as indicated by arrow R3, to align a fourth control element indicator 240d with a corresponding sixth housing indicator 242f, labeled “LOCK”. Accordingly, the coaption control 270 is (releasably) secured in position, reducing the likelihood that the electrode device 280 (FIG. 3H) will move as it is sealing the PFO. At this point, the arrowhead of the fifth directional indicator 241e directs the practitioner to the sixth directional indicator 241f, labeled “TREAT”.

With the electrode device 280 in the position shown in FIG. 3H, the practitioner can treat the patient by applying electrical energy (e.g., a varying electrical current) to the electrode device 280. In a particular embodiment, the energy is applied by operating a foot switch (not shown). In representative embodiments, electrical energy is applied to an electrode device 280 having a diameter in the range of about 3 mm to about 30 mm, at a frequency in the range of about 100 KHz to about 5 MHz for a period of up to 10 minutes (e.g., in a particular embodiment, from about 30-120 seconds). The energy can be provided at a rate in the range of about 10 Watts to about 500 Watts, and in a particular embodiment in the range of about 40-50 Watts. Different sizes and shapes of the PFO (or other tissue defect) will typically determine the particular electrode device size and/or energy delivery parameters. For example, the electrode device 280 can have a diameter of from about 7 mm to about 20 mm, and in a particular embodiment, about 9 mm. In a particular embodiment, the electrical energy can be applied to a 9 mm diameter electrode device at a frequency of about 450 KHz, for about 5 seconds, at a rate of from about 300 Watts to about 400 Watts. The electrical energy can be applied with a sinusoidal waveform, square waveform, or another periodic waveform shape, generally with a crest factor of from about one to about fifteen. RF energy provided to the electrode device 280 is received by the adjacent tissue so as to heat both the primum 107 and the secundum 108. The heat can at least partially fuse, glue, cement, or otherwise seal, join or connect the primum 107 and the secundum 108 together, forming a seal 118 that partially or completely closes the PFO tunnel 112 between the left atrium 102 and the right atrium 101.

FIG. 31 illustrates the second controller 260b with the controls 267-270 positioned for treating the patient by applying electrical current to the electrode device 280 described above with reference to FIG. 3H. In this configuration, the fourth control element indicator 240d is aligned with the sixth housing indicator 242f and the practitioner seals the patient's PFO.

After the practitioner has sealed the patient's PFO, the seventh directional indicator 241g labeled “REVERSE STEPS” directs the practitioner to reverse the previous steps. Accordingly, the practitioner begins by unlocking the coaption control 270, rotating it counterclockwise as indicated by arrow R4, and sliding it to the left as indicated by arrow T5 to rejoin the two portions 271, 272 of the second carriage 265. The practitioner next rotates the electrode deployment control 269 counterclockwise as indicated by arrow R5 and translates the electrode deployment control 269 to the right as indicated by arrow T6, thus stowing the electrode device and moving it back from the left atrium to the right atrium. The practitioner then slides the penetrating guidewire control 268 from left to right, as indicated by arrow T7 to move the penetrating guidewire from the left atrium to the right atrium. Finally, the practitioner rotates the catheter bend control 267 counterclockwise as indicated by arrow R6 to restow the positioning catheter 230b (FIG. 3H), allowing the deployment catheter 230c (FIG. 3H) to be withdrawn from the patient's body.

FIG. 3J illustrates the patient's septum 106 after the tissue fusing and/or sealing process has been completed. A small residual opening 119 may remain in the seal 118 as a result of withdrawing the electrode catheter 230c and penetrating guidewire 250d (FIG. 3H) back through the septum 106 from the left atrium 102 to the right atrium 101. The residual opening 119 is typically very small (e.g., on the order of one millimeter) and is expected to close quickly as a result of the body's normal healing process. The practitioner then withdraws the delivery catheter 230a from the patient's body. In other cases in which the seal 118 may initially be incomplete for other reasons, it is also expected that the seal will be sufficient to allow the body's normal healing processes to complete the closure, generally in a short period of time.

FIG. 4 is a partially schematic, cutaway illustration of the second controller 260b, as seen from the bottom, with a lower portion of the housing 261b removed. FIG. 4 illustrates several details of the internal structure of the second controller 260b in accordance with a particular embodiment. As shown in FIG. 4, the catheter bend control 267 can include a spiral slot SL that engages with a pin (not visible in FIG. 4) carried by the first carriage 264. The housing 261b can include supports 273 that align and support sliding movement of the components as they move axially within the delivery catheter 230a. The supports 273 can also have integrated hemostasis valves to prevent an unintended flow of bodily fluids into the housing 261b, and to direct flushing fluids through the delivery catheter 230a. The housing 261b can also include one or more springs 274 to bias particular control elements to particular settings. For example, the springs 274 can bias the first and second portions 271, 272 of the second carriage 265 to be positioned together, as shown in FIG. 4. The force provided by the springs 274 provides the compressing or coapting force on the primum and secundum, as discussed previously with respect to FIG. 3G.

In other embodiments, the second controller 260b can include other arrangements of particular control elements and associated linkages with the devices that they control. In any of these embodiments, the arrangement of the control elements can be generally similar to that described above with reference to FIGS. 3C-31 to provide an intuitive, logical, sequential, and/or ordered arrangement for the practitioner. It is expected that this arrangement can simplify the practitioner's task during a tissue sealing operation, thus increasing the efficacy and/or speed with which the practitioner can complete the operation, e.g., by reducing the amount of time the practitioner spends identifying and/or confirming the order in which the control elements are to be manipulated.

From the foregoing, it will be appreciated that specific embodiments of the invention have been described herein for purposes of illustration, but that various modifications may be made without deviating from the invention. For example, the housing indicators, control element indicators, and/or directional indicators can have configurations, shapes and/or legends other than those specifically shown and described above. The electrodes and self-centering guidewire can have configurations other than those specifically shown and described above, including, but not limited to, those described in co-pending U.S. application Ser. No. ______ (Attorney Docket No. 57120.8016US1). Certain aspects of the invention described in the context of particular embodiments may be combined or eliminated in other embodiments. For example, while certain embodiments were described above in the context of a clockwise arrangement of control elements for sealing a patient's PFO, in other embodiments, the order can be counterclockwise. Further, while advantages associated with certain embodiments have been described in the context of those embodiments, other embodiments may also exhibit such an advantages. Not all embodiments need necessarily exhibit such advantages to fall within the scope of the invention. Accordingly, the disclosure can include other embodiments not shown or described above.

I/We claim:

1. A patient treatment system, comprising:
a catheter carrying multiple active elements; and
a controller connected to the catheter, the controller including:
a housing having directional indicators; and
multiple control elements coupled to the multiple active elements, with individual control elements movable relative to the housing to control motion of the active elements, wherein the multiple control elements are positioned so that manipulation of the multiple control elements in a first order that is clockwise or counterclockwise as identified by the directional indicators moves the multiple active elements in a first manner, and manipulation of the multiple control elements in a second order opposite the first order moves the multiple active elements in a second manner opposite the first manner.

2. The system of claim 1 wherein the first order is a generally clockwise order and the second order is a generally counterclockwise order.

3. The system of claim 1 wherein the directional indicators include a linear directional indicator directing motion of the control elements from right to left along a lower portion of the housing, another linear directional indicator directing motion of the control elements from left to right along an upper portion of the housing, and still another directional indicator positioned between the linear directional indicators and directing rotation of at least one of the multiple control elements in a clockwise direction.

4. The system of claim 1 wherein at least some of the control elements are positioned in a serial, sequential order according to which the control elements are to be manipulated.

5. The system of claim 1 wherein at least one of the control elements has a control element indicator, and wherein the housing has a corresponding housing indicator, and wherein the control element indicator aligns with the corresponding housing indicator when a corresponding active element is positioned to be controlled by the corresponding control element.

6. The system of claim 1 wherein at least one of the control elements has a control element indicator, and wherein the housing has a corresponding housing indicator, and wherein the control element indicator aligns with the corresponding housing indicator only when a corresponding active element is positioned to be controlled by the corresponding control element.

7. The system of claim 1 wherein at least one of the multiple control elements is both slideable and rotatable relative to the housing.

8. The system of claim 7 wherein the at least one control element has a control element indicator, and wherein the housing includes three corresponding housing indicators, and wherein the at least one control element is slideable from a first position with the control element indicator aligned with a first one of the housing indicators, and to a second position with the control element indicator aligned with a second one of the housing indicators, and wherein the at least one control element is rotatable from the second position with the control element indicator aligned with the second housing indicator, to a third position with the control element indicator aligned with a third one of the housing indicators.

9. The system of claim 1, further comprising a deployable, RF electrode coupled to a power supply to at least partially seal cardiac tissue when activated, and wherein the at least one control element includes a control element operatively coupled to the RF electrode to deploy the RF electrode into position.

10. A patient treatment system, comprising:
a positioning catheter having multiple active elements, including:
a delivery catheter movably carried in the positioning catheter;
an electrode catheter movably carried in the delivery catheter;
a tissue penetrating guidewire movably carried in the electrode catheter;
an electrode carried by the electrode catheter; and
an electrode actuator connected to the electrode and movably carried in the electrode catheter; and
a controller connected to the positioning catheter, the controller including:
a housing having directional indicators identifying a clockwise sequence; and
a plurality of control elements, including:
a catheter bend control coupled to the delivery catheter and reversibly rotatable between a first position with the delivery catheter housed within the positioning catheter, and a second position with the delivery catheter bent so as to at least partially exit the positioning catheter;
a penetrating guidewire control coupled to the penetrating guidewire, positioned adjacent to the catheter bend control, and reversibly slideable between a third position in which the penetrating guidewire is housed in the electrode catheter and a fourth position in which the penetrating guidewire is advanced outwardly from the electrode catheter from the third position;
a coaption control coupled to the electrode, positioned adjacent to the penetrating guidewire control, and reversibly rotatable between a fifth position in which the electrode is spaced a first distance from the positioning catheter and a sixth position in which the electrode is spaced a second distance less than the first distance from the positioning catheter; and
an electrode deployment control coupled to the electrode actuator, positioned adjacent to the coaption control, and reversibly slideable between a seventh position in which the electrode is housed within the electrode catheter and an eighth position in which the electrode is deployed from the electrode catheter, the electrode deployment control being rotatable between a ninth position in which the electrode has a collapsed configuration and a tenth position in which the electrode has an erected configuration; and
wherein at least one of the controls includes an indicator that aligns with a corresponding indicator of the housing only when the active element to which it is coupled is positioned for movement.

11. The system of claim 10 wherein at least one of the plurality of control elements is both slideable and rotatable relative to the housing.

12. The system of claim 11 wherein the at least one control element has a control element indicator, and wherein the housing includes three corresponding housing indicators, and wherein the at least one control element is slideable from a first position with the control element indicator aligned with a first one of the housing indicators, to a second position with the control element indicator aligned with a second one of the housing indicators, and wherein the at least one control element is rotatable from the second position with the control element indicator aligned with the second housing indicator, to a third position with the control element indicator aligned with a third one of the housing indicators.

13. A method for treating a patient, comprising:
introducing a catheter into a patient, the catheter carrying multiple active elements; and
operating a controller connected to the catheter to move the active elements, wherein the controller includes a housing having directional indicators and multiple control elements, and wherein operating includes:
manipulating the multiple control elements in a first order that is clockwise or counterclockwise as identified by the directional indicators to move the at least one active element in a first manner; and
manipulating the multiple control elements in a second order opposite the first order to move the at least one active element in a second manner opposite the first manner.

14. The method of claim 13 wherein manipulating the control elements includes:
manipulating a first control element to move a first one of the active elements in a first manner and to align an indicator carried by a second control element with a corresponding indicator carried by the housing; and
manipulating the second control element when the indicator carried by the second control element aligns with the corresponding indicator carried by the housing to move the first active element or a second active element in a second manner different than the first.

15. The method of claim 13 wherein manipulating the control elements in a first order includes manipulating the elements in a generally clockwise order, and wherein manipulating the control elements in a second order includes manipulating the control elements in a generally counterclockwise order.

16. The method of claim 13 wherein manipulating the multiple control elements includes both sliding and rotating at least one of the control elements relative to the housing.

17. The method of claim 16 wherein the at least one control element has a control element indicator, and wherein the housing includes three corresponding housing indicators, and wherein manipulating the at least one control element includes sliding the at least one control element from a first position with the control element indicator aligned with the first housing indicator, to a second position with the control element indicator aligned with a second housing indicator, and rotating the at least one control element from the second position with the control element indicator aligned with the second housing indicator, to a third position with the control element indicator aligned with a third housing indicator.

18. The method of claim 13 wherein manipulating the control elements in a first order includes:
operating a penetrating guidewire control element to direct a tissue-penetrating guidewire through a patient's interatrial septum;
operating an electrode deployment control element in a first manner to move a tissue-sealing electrode along the tissue-penetrating guidewire from the patient's right atrium to the patient's left atrium;
operating the electrode deployment control element in a second manner different than the first manner to expand the tissue-sealing electrode in the patient's left atrium; and wherein the method further comprises:
at least partially sealing a PFO in the patient's interatrial septum while the tissue-sealing electrode is in the left atrium.

19. The method of claim 13 wherein the catheter includes:
a positioning catheter;
a delivery catheter movably carried in the positioning catheter;
an electrode catheter movably carried in the delivery catheter;
a tissue penetrating guidewire movably carried in the electrode catheter;
an electrode carried by the electrode catheter;
an electrode actuator connected to the electrode and movably carried in the electrode catheter; and wherein the multiple control elements include:
a catheter bend control coupled to the delivery catheter;
a penetrating guidewire control coupled to the penetrating guidewire and positioned adjacent to the catheter bend control;
a coaption control coupled to the electrode and positioned adjacent to the penetrating guidewire control; and
an electrode deployment control coupled to the electrode actuator and positioned adjacent to the coaption control; and wherein manipulating the control elements in a first order includes:
reversibly rotating the catheter bend control between a first position with the delivery catheter housed within the positioning catheter, and a second position with the delivery catheter bent so as to at least partially exit the positioning catheter;
reversibly sliding the penetrating guidewire control between a third position in which the penetrating guidewire is housed in the electrode catheter and a fourth position in which the penetrating guidewire is advanced outwardly from the electrode catheter from the third position;
reversibly sliding the electrode deployment control between a fifth position with the electrode in the patient's right atrium and a sixth position with the electrode in the patient's left atrium;
reversibly rotating the electrode deployment control between a seventh position in which the electrode has a collapsed configuration and an eighth position in which electrode has an expanded configuration;
reversibly sliding the electrode deployment control between a ninth position with the electrode in the patient's right atrium and spaced a first distance from the positioning catheter, and a tenth position in which the electrode is spaced a second distance less than the first distance from the positioning catheter to compress the patient's secundum and primum between the electrode and the positioning catheter; and wherein the method further comprises:
at least partially sealing a PFO tunnel between the secundum and the primum by applying RF energy to the electrode when the electrode is positioned in the left atrium and is forcing the secundum and primum toward the positioning catheter.
